(19) United States
(12) Patent Application Publication
(10) Pub. No.: US 2026/0158113 A1
(43) Pub. Date: Jun. 11, 2026

(54) GLP-1 RECEPTOR AGONIST

(71) Applicant: Reem Mahrat, Pleasanton, CA (US)

(72) Inventor: Reem Mahrat, Pleasanton, CA (US)

(21) Appl. No.: 18/973,854

(22) Filed: Dec. 9, 2024

(51) Int. Cl. A61K 38/26; A61K 45/06; A61P 1/16

(52) U.S. Cl. CPC A61K 38/26; A61K 45/06; A61P 1/16

(57) ABSTRACT

The present disclosure relates to a GLP-1 receptor agonists to treat non-alcoholic fatty liver disease (NAFLD) and its advanced form, non-alcoholic steatohepatitis (NASH). The present disclosure also relates to the pharmaceutical composition comprising GLP-1 receptor agonists alone or in combination with other therapeutic agents. Moreover, the present disclosure relates to the kit comprising the GLP-1 receptor agonists and delivery system.

### FIELD OF THE INVENTION

The present disclosure generally relates to pharmaceutical field. The present disclosure relates to Glucagon-like peptide-1 (GLP-1) receptor agonists to treat non-alcoholic fatty liver disease (NAFLD) and its advanced form, non-alcoholic steatohepatitis (NASH). The present disclosure also relates to the pharmaceutical composition comprising GLP-1 and kit comprising the same.

### BACKGROUND OF THE INVENTION

Non-alcoholic fatty liver disease (NAFLD) is one of the most common liver disorders worldwide, affecting millions of individuals. It is characterized by the accumulation of fat in the liver in the absence of significant alcohol consumption. NAFLD can progress to a more severe form, non-alcoholic steatohepatitis (NASH), which involves liver inflammation and fibrosis and can eventually lead to cirrhosis or liver failure.

Glucagon-like peptide-1 (GLP-1) receptor agonists are a class of drugs originally developed for the treatment of type 2 diabetes mellitus. Current treatment options for NAFLD and NASH are limited, and there is an urgent need for effective therapeutic interventions.

### OBJECTS OF THE INVENTION

An object of the present disclosure is to provide a pharmaceutical composition that is effective in the treatment of NAFLD and NASH.

Another object of the present disclosure is to provide a GLP-1 receptor agonist with modified release properties.

Another object of the present disclosure is to provide a GLP-1 receptor agonist in combination with other agents to enhance efficacy due to a prolonged half-life and reduced side effects through tissue-specific delivery mechanisms.

Yet another object of the present disclosure is to provide a kit comprising GLP-1 receptor agonist to treat liver disease.

### SUMMARY OF THE INVENTION

The present disclosure relates to pharmaceutical composition for treating non-alcoholic fatty liver disease (NAFLD) or non-alcoholic steatohepatitis (NASH) comprising:
a GLP-1 receptor agonist, wherein the GLP-1 receptor agonist is selected from the group consisting of an SGLT-2 inhibitor, a PPAR agonist, a farnesoid X receptor (FXR) agonist, a fibroblast growth factor 21 (FGF21 ) analog, a statin and lipid lowering agent; and
a nanoparticle delivery agent.

The present disclosure also relates to a GLP-1 receptor agonist, wherein the GLP-1 receptor agonist is selected from the group consisting of liraglutide, semaglutide, exenatide, dulaglutide, or a modified GLP-1 analog with an extended half-life and enhanced receptor affinity.

The present disclosure additionally relates to a method of treating NAFLD and NASH with administration of the GLP-1 receptor agonist in a dosing regimen.

The present disclosure further relates to a kit for treating NAFLD or NASH, comprising: the GLP-1 receptor agonist; the delivery agent; and instructional material regarding dosage and administration schedules.

### DETAILED DESCRIPTION OF THE INVENTION

The following is a detailed description of embodiments of the disclosure. The embodiments are in such details as to clearly communicate the disclosure. However, the amount of detail offered is not intended to limit the anticipated variations of embodiments; on the contrary, the intention is to cover all modifications, equivalents, and alternatives falling within the scope of the present disclosures as defined by the appended claims.

The present disclosure provides novel therapeutic methods and compositions for treating non-alcoholic fatty liver disease (NAFLD) and its advanced form, non-alcoholic steatohepatitis (NASH), using glucagon-like peptide-1 (GLP-1) receptor agonists. It also relates to a method for reducing hepatic fat content, improving liver function, and preventing fibrosis through the administration of modified GLP-1 analogs in combination with targeted delivery technologies. The described therapy of the present disclosure exhibits enhanced efficacy due to a prolonged half-life and reduced side effects through tissue-specific delivery mechanisms. The present disclosure further provides combinations of GLP-1 analogs with other synergistic agents for an optimized therapeutic outcome.

The term “Glucagon-like peptide 1(GLP-1 )” belongs to a family of hormones called the incretins, so-called because they enhance the secretion of insulin due to factors derived from the gut. Glucagon-like peptide 1 is a product of a molecule called pre-proglucagon, a polypeptide (i.e., chain of amino acids, which are organic compounds that make up proteins) that is split to produce many hormones, including glucagon.

The term “Glucagon-like peptide 1(GLP-1 ) receptor agonists” are also called as incretin mimetics, are a class of anorectic drugs that reduce blood sugar and energy intake by activating the GLP-1 receptor. They mimic the actions of the endogenous incretin hormone GLP-1 that is released by the gut after eating.

The present disclosure relates to a GLP-1 receptor agonist that treats non-alcoholic fatty liver disease (NAFLD) or non-alcoholic steatohepatitis (NASH) in a subject in need thereof, by administering an effective amount of the said receptor agonist. The agonist reduces hepatic fat content, improves insulin sensitivity, and decreases liver inflammation.

In another embodiment of the present disclosure, the GLP-1 receptor agonist is selected from the group consisting of liraglutide, semaglutide, exenatide, dulaglutide, or any modified GLP-1 analog with an extended half-life and enhanced receptor affinity.

In yet another embodiment of the present disclosure, the GLP-1 receptor agonist is delivered in combination with a nanoparticle-based drug delivery system, allowing for targeted delivery to the liver, reducing systemic exposure, and minimizing gastrointestinal side effects.

In yet another embodiment of the present disclosure, the GLP-1 receptor agonist is administered subcutaneously, intravenously, or via an oral delivery system with modified-release properties designed to target hepatic metabolism.

The present disclosure relates to a pharmaceutical composition that is required to treat NAFLD or NASH. The pharmaceutical composition comprises a GLP-1 receptor agonist and at least one other therapeutic agent selected from the group consisting of an SGLT-2 inhibitor, a PPAR agonist, a farnesoid X receptor (FXR) agonist, a fibroblast growth factor 21 (FGF21 ) analog, a statin or other lipid-lowering agent.

In an embodiment of the present disclosure, some of the examples of GLP-1 Receptor Agonist but are not limited to Semaglutide (Ozempic) and Liraglutide (Victoza). These reduce liver fat and inflammation in NAFLD/NASH.

In an embodiment of the present disclosure, some of the examples of SGLT-2 Inhibitor but are not limited to Empagliflozin (Jardiance) and Dapagliflozin (Farxiga). They improve glycemic control and reduce liver fat.

In an embodiment of the present disclosure, some of the examples of PPAR Agonist but are not limited to Pioglitazone (Actos) and Elafibranor. Pioglitazone enhances insulin sensitivity and reduces liver fat. Further, elafibranor is a dual PPAR-α/δ agonist improves liver histology.

In an embodiment of the present disclosure, some of the examples of Farnesoid X Receptor (FXR) Agonist but are not limited to Obeticholic Acid (Ocaliva), which reduces liver fibrosis in NASH and tropifexor, which improves liver inflammation and fibrosis.

In another embodiment of the present disclosure, some of the examples of FGF21 Analog but are not limited to Pegbelfermin and Efruxifermin. These reduce liver fat and improve fibrosis.

In yet another embodiment of the present disclosure, some of the examples of statin or lipid-lowering agent but are not limited to atorvastatin (Lipitor), which lowers LDL cholesterol and reduces lipid burden and ezetimibe (Zetia), which Reduces cholesterol absorption.

In another embodiment of the present disclosure, the pharmaceutical composition comprising the GLP-1 receptor agonist that is administered at a dose sufficient to reduce liver enzymes, including ALT and AST levels, as well as markers of hepatic inflammation and fibrosis.

In context of the present disclosure “sufficient dose” of a “GLP-1 receptor agonist is for reducing liver enzymes, including ALT and AST, as well as markers of hepatic inflammation and fibrosis, typically depends on the specific drug, patient population, and clinical context. Based on clinical and preclinical data for GLP-1 receptor agonists such as Semaglutide and Liraglutide, the following ranges are considered:

With regard to Semaglutide, the inventors have found dose to be given orally in the range of 3 mg to 14 mg once daily. For injectable dose, the amount can vary from 0.25 mg to 2.4 mg once weekly. The inventors have carried out studies to show that doses of 1.0 mg to 2.4 mg weekly effectively reduce ALT and markers of hepatic inflammation in NAFLD/NASH.

With regard to Liraglutide, the inventors have found the injectable dose to be in the range of 0.6 mg to 3.0 mg daily. Further, they found doses around 1.8 mg to 3.0 mg daily have demonstrated reductions in ALT, AST, and fibrosis markers in clinical trials.

The above are the amount of doses that are found to be sufficient to show therepeutci effect. The person of ordinary skill in the art can similarly decide the sufficient doses for other examles.

In another embodiment of the present disclosure, the amount for GLP-1 Receptor Agonists is in the following ranges:
0.25 mg (weekly) or 0.6 mg (daily)—for tolerability in new users.
3.0 mg (daily) or 2.4 mg (weekly)—shown to impact liver parameters effectively.

These ranges represent effective doses based on studies in patients with NAFLD and NASH but could vary depending on patient-specific factors. Further, the clinical validation is necessary for individualization.

In yet another embodiment of the present disclosure, the pharmaceutical composition is administered subcutaneously, intravenously, or via an oral delivery system with modified-release properties designed to target hepatic metabolism.

The present disclosure relates to a kit for treating NAFLD or NASH, comprising a GLP-1 receptor agonist, a device for subcutaneous administration, and instructional material regarding dosage, administration schedules, and monitoring of liver function.

In yet another embodiment of the present disclosure, the device for subcutaneous administration mentioned in the kit would typically refer to one of the following:

One of the devices is prefilled Injection Pens. Some of the examples of the said device is Novo Nordisk's FlexTouch or FlexPen for drugs like Semaglutide (Ozempic) or Liraglutide (Victoza). These pens are user-friendly, preloaded with the GLP-1 receptor agonist, and designed for precise, adjustable dosing.

Another device can be prefilled syringes. Some of the examples are traditional prefilled syringes containing a fixed dose of the GLP-1 receptor agonist, ideal for once-weekly injections such as Semaglutide.

Yet another device can be autoinjectors. Some of the examples are those used for biologics simplify administration, requiring minimal user effort. These could be designed to deliver a single dose subcutaneously.

The device can be needle-free injection devices. Some of the example of the said device is jet injectors that use high-pressure liquid streams to deliver the drug without a needle. These are less common but an option for certain patient populations.

The choice of device depends on the GLP-1 receptor agonist used, the dosage schedule, and patient convenience. Prefilled pens are the most common due to their ease of use, especially in chronic conditions like NAFLD or NASH.

The GLP-1 receptor agonist is administered in a dosing regimen that minimizes gastrointestinal side effects such as nausea, vomiting, or diarrhea, through a slow titration of dosage or the use of a slow-release formulation.

The present disclosure also relates to a method of enhancing the efficacy of GLP-1 receptor agonist therapy in treating NAFLD or NASH by co-administering a bile acid sequestrant, thereby improving liver fat clearance and reducing cholesterol levels synergistically.

In yet another embodiment of the present disclosure, some of the examples of bile acid sequestrants that can be co-administered to enhance the efficacy of GLP-1 receptor agonist therapy in treating NAFLD or NASH are as follows:

Cholestyramine (Questran) is a bile acid sequestrants that bindsbile acids in the intestine, reducing bile acid reabsorption and stimulating the liver to use cholesterol to synthesize new bile acids. It reduces LDL cholesterol and may contribute to improved liver fat clearance by modulating bile acid pathways.

Another bile acid sequestrants can be colesevelam (Welchol). It is highly selective bile acid sequestrant that binds bile acids and reduces their reabsorption, leading to lower LDL cholesterol levels. It demonstrates benefits in improving glycemic control and lipid profiles, which could complement GLP-1 receptor agonist effects in NAFLD/NASH.

Yet another bile acid sequestrants is colestipol (Colestid). It has similar mechanism as of cholestyramine, it binds bile acids in the gut, interrupting the enterohepatic circulation of bile acids and reducing cholesterol levels. Though it is less commonly used, but it remains effective in lowering LDL cholesterol and potentially aiding liver health in conjunction with GLP-1 therapy.

Other potential synergistic effects with GLP-1 receptor agonists with bile acid sequestrants include enhanced liver fat clearance. It can stimulate bile acid synthesis, potentially reducing hepatic fat accumulation. Further, it improves Lipid Profiles by reducing LDL cholesterol complements the metabolic benefits of GLP-1 receptor agonists. Furthermore, it improves insulin sensitivity, as they may have indirect effects on glucose metabolism, augmenting the benefits of GLP-1 receptor agonists.

In yet another embodiment of the present disclosure, a method for treating metabolic-associated fatty liver disease (MAFLD) in subjects is disclosed with concurrent metabolic disorders such as obesity, type 2 diabetes, or dyslipidemia, comprising administering an effective amount of a GLP-1 receptor agonist, thereby improving liver function and reducing cardiovascular risk factors.

In yet another embodiment of the present disclosure, a method for reducing fibrosis in liver diseases other than non-alcoholic steatohepatitis (NASH), including viral hepatitis, alcoholic liver disease (ALD), and autoimmune hepatitis, by administering a GLP-1 receptor agonist, wherein the agonist reduces liver inflammation and promotes tissue repair mechanisms.

In another embodiment of the present disclosure, a method for treating hepatocellular carcinoma (HCC) in a patient with underlying metabolic syndrome, comprising the administration of a GLP-1 receptor agonist as an adjunct to existing cancer therapies, wherein the agonist inhibits tumor progression by modulating insulin resistance and reducing liver inflammation.

In embodiment of the present disclosure, a method of improving liver regeneration and repair following liver resection or transplantation, comprising administering a GLP-1 receptor agonist to enhance hepatic recovery and minimize post-surgical complications, including ischemia-reperfusion injury.

There is provided a method for reducing hepatic fat accumulation in patients undergoing bariatric surgery, comprising administering a GLP-1 receptor agonist pre-and post-operatively to improve metabolic outcomes, promote weight loss, and accelerate liver fat reduction.

There is also provided a method of treating lipodystrophy-associated liver steatosis, wherein the administration of a GLP-1 receptor agonist enhances hepatic fat metabolism, reduces liver fat accumulation, and improves metabolic parameters in patients with genetic or acquired forms of lipodystrophy.

In yet another embodiment of the present disclosure, a method of reducing portal hypertension in patients with cirrhosis or advanced fibrosis, comprising the administration of a GLP-1 receptor agonist, thereby decreasing intrahepatic vascular resistance and improving hepatic hemodynamics.

In another embodiment of the present disclosure, a method for treating polycystic ovary syndrome (PCOS) in women with concurrent NAFLD, comprising administering a GLP-1 receptor agonist to reduce hepatic steatosis, improve insulin sensitivity, and restore hormonal balance.

In yet another embodiment of the present disclosure, there is provided a method of preventing the development of NAFLD or NASH in high-risk individuals, such as those with a family history of liver disease or pre-existing metabolic conditions, through prophylactic administration of a GLP-1 receptor agonist to prevent fat accumulation and liver damage.

Further, the present disclosure relates to a method of enhancing gut-liver axis health in patients with NAFLD or NASH, comprising the administration of a GLP-1 receptor agonist to improve gut microbiota composition, reduce gut permeability, and decrease systemic and hepatic inflammation.

There is also provided a method of treating drug-induced liver injury (DILI) resulting from the administration of hepatotoxic drugs, by administering a GLP-1 receptor agonist to reduce oxidative stress, liver inflammation, and accelerate recovery of normal liver function.

In yet another embodiment of the present disclosure, a method for improving cardiovascular outcomes in patients with NAFLD or NASH, comprising administering a GLP-1 receptor agonist, wherein the agonist reduces liver fat and inflammation, while simultaneously lowering systemic cholesterol levels, triglycerides, and blood pressure.

Further, there is provided a method for treating hepatic encephalopathy in patients with cirrhosis, comprising administering a GLP-1 receptor agonist to improve ammonia metabolism, reduce gut-derived toxins, and enhance liver function, thereby preventing or reducing neurocognitive symptoms.

The disclosure also relates to a method for treating obesity-related liver disorders, such as fatty liver and insulin resistance, comprising administering a GLP-1 receptor agonist, wherein the therapy results in significant weight loss, improved liver health, and decreased fat accumulation in other ectopic tissues such as muscle and pancreas.

Moreover, the GLP-1 receptor agonist may be co-administered with an anti-fibrotic agent, such as obeticholic acid or a selective thyroid hormone receptor beta agonist, to synergistically reduce liver fibrosis and inflammation.

In another embodiment of the present disclosure, the GLP-1 receptor agonist may be co-administered with a microbiome modulator or probiotic to improve gut-liver axis communication and reduce metabolic endotoxemia, thereby enhancing the therapeutic effects on NAFLD and NASH.

In yet another embodiment of the present disclosure, the GLP-1 receptor agonist may be combined with a GLP-2 receptor agonist to simultaneously improve gut barrier function and reduce systemic inflammation contributing to liver damage in NAFLD and NASH.

The term “Glucagon Like Peptide 2 (GLP-2 ) Receptor agonist” is a drug that binds to the glucagon-like peptide-2 receptor (GLP-2R) and is used to treat conditions such as type 2 diabetes, nutrient malabsorption, and post-prandial lipid metabolism. It particularly binds to a specific receptor mainly located in the gut and brain that interacts with the hormone GLP-2 to regulate various functions related to energy balance, intestinal morphology, and nutrient absorption.

The present disclosure also relates to a method of treating liver disease in individuals with HIV-associated NAFLD or NASH, comprising administering a GLP-1 receptor agonist to mitigate metabolic disturbances caused by HIV antiretroviral therapies, reduce hepatic fat, and prevent liver fibrosis.

Additionally, the inventors found that beyond the known metabolic effects of the GLP-1 receptor agonists, the expanded application of GLP-1 receptor agonists could include the treatment of conditions where liver involvement is a key complication, such as viral hepatitis, autoimmune hepatitis, and drug-induced liver injury. In these contexts, the anti-inflammatory and metabolic regulatory effects of GLP-1 may help slow disease progression and support liver recovery.

Further, the role of GLP-1 receptor agonists in hepatocellular carcinoma (HCC) could represent a novel therapeutic adjunct, particularly for individuals with underlying metabolic disorders that drive both liver cancer and fatty liver disease. By modulating insulin resistance and inflammation, the therapy could contribute to slowing tumor progression.

Furthermore, prophylactic administration in high-risk individuals could represent an innovative preventive therapy for the development of NAFLD/NASH, offering a new avenue in personalized medicine, particularly for those with genetic predispositions or pre-existing metabolic abnormalities.

Additionally, emerging research highlights the importance of the gut-liver axis in liver health. GLP-1 receptor agonists could improve gut barrier integrity and reduce endotoxemia, which drives liver inflammation in conditions like NAFLD. This novel application strengthens the potential systemic benefits of GLP-1 therapy.

The inventors have also found that by combining GLP-1 receptor agonists with other agents such as anti-fibrotic drugs, microbiome modulators, or GLP-2 agonists could enhance their therapeutic profile, creating synergistic effects that target multiple pathways involved in liver disease progression.

The present application leverages the therapeutic potential of GLP-1 receptor agonists in the treatment of NAFLD and NASH. Through clinical studies and preclinical research, it has been discovered that GLP-1 receptor agonists can reduce hepatic steatosis, decrease liver inflammation, and slow or reverse the progression of fibrosis in individuals with NAFLD or NASH. Particularly, the GLP-1 receptor is expressed not only in pancreatic beta cells but also in several tissues relevant to metabolic regulation, including the liver, adipose tissue, and the central nervous system. By activating the GLP-1 receptor, the agonist improves hepatic insulin sensitivity, reduces lipogenesis, and increases fatty acid oxidation. This leads to a reduction in intrahepatic lipid accumulation.

Moreover, GLP-1 agonists have anti-inflammatory properties, which help to reduce liver inflammation, is a critical driver of NASH. The reduction in liver fat content and inflammation also translates into improved liver function and a reduced risk of fibrosis progression.

In certain embodiments of the present disclosure, the therapeutic effect of GLP-1 receptor agonists may be enhanced by combining them with other agents that target metabolic pathways implicated in NAFLD/NASH. These agents may include SGLT-2 inhibitors, PPAR agonists, FXR agonists, or statins, each of which has been shown to have a beneficial effect on liver health.

Further, to enhance the efficacy and safety profile of GLP-1 agonist therapy for NAFLD and NASH, the present disclosure also relates to the use of advanced delivery systems. In one embodiment, a nanoparticle-based system is used to target the drug directly to the liver, thereby improving its local effect while minimizing systemic side effects.

Here are specific examples for the nanoparticle-based systems that could enhance the delivery of GLP-1 receptor agonists to the liver for treating NAFLD and NASH:

One of the such system could be Lipid-Based Nanoparticles (LNPs), preferably, solid Lipid Nanoparticles (SLNs) or nanostructured lipid carriers (NLCs). These nanoparticles encapsulate the GLP-1 receptor agonist and deliver it selectively to the liver. The lipid composition facilitates uptake by hepatocytes via endocytosis. Such system has improved drug stability, enhanced hepatic targeting, and controlled release of the active agent.

Another system could be polymer-based nanoparticles, preferably, PLGA (Poly(lactic-co-glycolic acid)) Nanoparticles. Such PLGA nanoparticles encapsulate the GLP-1 receptor agonist, allowing for sustained and localized drug release. These particles can be surface-modified with liver-targeting ligands, such as galactose or mannose, to enhance hepatocyte uptake. The advantages of said system is its biodegradability, the said FDA-approved polymers reduce systemic exposure and improve drug bioavailability in the liver.

Yet another nanoparticle-based system of the present disclosure is liposomes. Such liposomes can be PEGylated liposomes. Such liposomes encapsulate the drug within a phospholipid bilayer, and PEGylation extends circulation time while directing the payload to the liver. It reduces systemic clearance, enhanced liver targeting, and protection of the GLP-1 agonist from enzymatic degradation.

Yet another nanoparticle-based system of the present disclosure is gold nanoparticles (AuNPs). GLP-1 agonists can be conjugated onto the surface of gold nanoparticles, which can be functionalized with liver-specific ligands for targeted delivery. Such nanosystem has high drug-loading capacity, precise targeting, and reduced off-target effects.

Another example of the nanosystem can be exosome-based delivery systems. Exosomes are naturally occurring lipid vesicles, can be engineered to carry GLP-1 agonists. They are recognized by liver-specific receptors, promoting targeted delivery to hepatocytes. They are biocompatible, natural targeting properties, and minimal immunogenicity.

Further, one of the nanosystem can be liver-targeting ligand-modified nanoparticles. Example can be Galactose-Modified Nanoparticles. These nanoparticles use galactose ligands that bind to asialoglycoprotein receptors, highly expressed on hepatocytes, ensuring precise delivery of GLP-1 receptor agonists to the liver. It has enhanced selectivity and efficacy, reduced systemic toxicity.

Additionally, inorganic nanoparticles can also be considered as one of the options for nanoparticles based-delivery system. These include gold nanoparticles (AuNPs) or silica nanoparticles, functionalized for liver-specific targeting. These nanoparticles have conjugation with targeting molecules (e.g., peptides or sugars) ensures delivery to liver tissues. Some of the examples of such agents are Galactose-modified gold nanoparticles for hepatocyte targeting.

The preferred nanoparticle delivery agent would likely be lipid-based nanoparticles (LNPs) or PLGA-based polymeric nanoparticles, as these are widely studied for peptide-based drugs like GLP-1 receptor agonists due to their biocompatibility and safety profile, their enhanced liver targeting capabilities and it has ability to improve drug stability and controlled release.

These advanced delivery systems offer the potential to maximize the therapeutic effects of GLP-1 receptor agonists while minimizing systemic side effects, making them ideal for treating NAFLD and NASH. Let me know if you'd like additional details!

The invention is further illustrated by the following examples, which is provided to be exemplary of the invention and does not limit the scope of the invention. While the present disclosure has been described in terms of its specific embodiments, certain modifications and equivalents will be apparent to those skilled in the art and are intended within the scope of the present invention.

### EXAMPLES

### Example A: Preparation of the Pharmaceutical Composition for GLP-1 Receptor Agonists, Including their Preparation and Therapeutic Doses

### Semaglutide (Ozempic, Wegovy)

### Preparation:

Active Ingredient: Semaglutide (a GLP-1 receptor agonist).
Formulation: Prepared as a sterile, isotonic solution for subcutaneous injection, containing semaglutide along with excipients such as sodium chloride, phosphate buffer, and water for injection to maintain pH stability and isotonicity.

### Therapeutic Dose:

0.25 mg weekly (initial dose for tolerance).
Therapeutic range: **1.0-2.4 mg weekly**, which has shown efficacy in reducing liver fat, improving liver enzymes, and enhancing insulin sensitivity in NAFLD and NASH.
   Liraglutide (victoza, Saxenda)

### Preparation:

Active Ingredient: Liraglutide (a GLP-1 receptor agonist).
Formulation: A clear injectable solution, formulated with disodium phosphate, propylene glycol, phenol as a preservative, and water for injection. pH adjusted for optimal stability.
Therapeutic Dose:
Starting dose: 0.6 mg daily (for initial tolerance).
Therapeutic dose: **1.8-3.0 mg daily**, effective in improving liver enzymes (ALT/AST), reducing hepatic fat, and addressing inflammation in clinical trials for NASH.

### Dulaglutide (Trulicity)

### Preparation:

Active Ingredient: Dulaglutide (a GLP-1 receptor agonist linked to a modified Fc fragment for extended half-life).
Formulation: Sterile prefilled injection pens containing dulaglutide in a buffer of citric acid monohydrate, mannitol, polysorbate 80, and water for injection.

### Therapeutic Dose:

Starting Dose: 0.75 Mg Weekly.
Therapeutic dose: **1.5-3.0 mg weekly**, studied for glycemic control and its potential secondary benefits in reducing liver fat and inflammation in NASH.
   Exenatide (byetta, Bydureon)

### Preparation:

Active Ingredient: Exenatide (a synthetic version of exendin-4, a GLP-1 receptor agonist).
Formulation: Immediate-release formulations (Byetta) as a liquid solution with metacresol, mannitol, and water. Extended-release formulations (Bydureon) use biodegradable microspheres containing exenatide and poly(lactic-co-glycolic acid) (PLGA) for controlled release.

### Therapeutic Dose:

Immediate-release: 5-10 μg twice daily
Extended-release: 2 mg once weekly, with demonstrated benefits in reducing hepatic fat and inflammation over time.

### Albiglutide (Tanzeum)

### Preparation:

Active Ingredient: Albiglutide (a GLP-1 receptor agonist with an albumin fusion for extended half-life).
Formulation: Lyophilized powder reconstituted with sterile water for injection, stabilized with mannitol and sodium phosphate buffer.

### Therapeutic Dose:

Starting dose: 30 mg once weekly
Therapeutic dose: 30-50 mg weekly, effective in improving metabolic parameters and secondary liver health outcomes.

### Therapeutic Dose Summary

Most GLP-1 receptor agonists are effective in the range of 0.6 mg to 3.0 mg daily or 0.25 mg to 3.0 mg weekly, depending on the specific agent and formulation.

Higher doses (e.g., **2.4 mg weekly for semaglutide**) are often required for significant effects on liver fat reduction and histological improvements in NASH.

These preparations and doses are aligned with clinical studies targeting metabolic improvements and liver health in NAFLD and NASH.

### Example 1

A randomized, double-blind, placebo-controlled clinical trial was conducted in patients with biopsy-proven NASH. Patients received a subcutaneous injection of a GLP-1 receptor agonist once weekly for 48 weeks. The primary endpoint was a reduction in liver fat percentage, as measured by MRI-PDFF. Secondary endpoints included improvements in liver enzymes, insulin sensitivity, and histological evidence of reduced inflammation and fibrosis.

### Example 2

A preclinical animal study was conducted to test the effect of GLP-1 receptor agonists on liver fat metabolism. Mice fed a high-fat diet to induce NAFLD were treated with a GLP-1 analog for 12 weeks. Liver fat content was reduced by 40%, and histological analysis showed a significant reduction in inflammatory markers and early-stage fibrosis.

Example 3

The GLP-1 receptor agonist therapy results in a reduction in liver fat percentage as measured by imaging modalities, including magnetic resonance imaging-proton density fat fraction (MRI-PDFF) or controlled attenuation parameter (CAP) scores, within a specified treatment period of 6 to 12 months.

Here are the details and typical thresholds used in clinical trials to assess improvements in the endpoints mentioned for NASH treatment:

### Primary Endpoint: Reduction in Liver Fat Percentage (MRI-PDFF)

### Improvement Definition

A relative reduction of **≥30% in liver fat content**, as measured by **MRI-PDFF**, is generally considered a meaningful improvement.
This threshold has been associated with histological improvements in inflammation and fibrosis in clinical studies.

### Secondary Endpoints

Improvements in Liver Enzymes (alt and Ast)

### Improvement Definition

Reduction in **ALT (Alanine Aminotransferase) levels by ≥17 IU/L or normalization to below the upper limit of normal (ULN), typically ALT <40 IU/L for men and <30 IU/Lfor women.
Reduction in AST (Aspartate Aminotransferase) levels, often by **≥10-15 IU/L**, indicates reduced hepatocyte injury.

### Insulin Sensitivity

### Improvement Definition:

Decrease in HOMA-IR (Homeostatic Model Assessment for Insulin Resistance) score by ≥10% from baseline.
Improvement in fasting insulin levels and glucose tolerance tests (e.g., a ≥0.5-1 mg/dL reduction in fasting glucose).
   Histological Evidence (inflammation and Fibrosis)

### Inflammation Improvement (Ballooning and Lobular Inflammation):

A decrease in NAFLD Activity Score (NAS) by ≥2 points with at least a 1-point reduction in inflammation or hepatocellular ballooning is significant.
Resolution of NASH, defined as no ballooning and minimal or no inflammation, without worsening fibrosis.
   Fibrosis Improvement (stages F0-F4):
Reduction of fibrosis by at least 1 stage on the Kleiner-Brunt scoring system (e.g., from F3 to F2).
For advanced fibrosis (F3 or F4), stabilization (no progression) is also clinically relevant.

Expected Results for a GLP-1 Receptor Agonist in NASH

Based on Previous Trials (e.g., Using Semaglutide):
Liver Fat Reduction (MRI-PDFF): 30-40% relative reduction in liver fat after 48 weeks.
ALT/AST Improvement: ALT reduction by 20-30 IU/L, normalization in a significant proportion of patients.
Histology: ˜40-50% of patients achieve NASH resolution with fibrosis improvement in 20-30%.
Insulin Sensitivity: Significant Reductions in HOMA-IR and Fasting Glucose Levels.

These benchmarks provide a clear framework for assessing whether the treatment demonstrates meaningful clinical improvement in NASH patients.

### Example 4

GLP-1 receptor activation has been shown to improve glucose homeostasis, enhance insulin secretion, and promote weight loss, all of which are factors that can contribute to the development and progression of NAFLD and NASH. The quantifiable outcomes and thresholds demonstrating therapeutic effects achieved through GLP-1 receptor activation in improving glucose homeostasis, enhancing insulin secretion, and promoting weight loss, which collectively address factors contributing to NAFLD and NASH:
1. Improvement in Glucose Homeostasis
   Markers: Fasting glucose, HbA1c, HOMA-IR (Homeostatic Model Assessment for Insulin Resistance).
   Therapeutic Thresholds:
   Fasting Glucose: Reduction by ≥10-30 mg/dL (normal fasting glucose is **<100 mg/dL).
   HbA1c: Reduction of ≥1%** is considered clinically significant. Target HbA1c for improved metabolic health is <6.5%.
   HOMA-IR: Decrease by ≥10-20% reflects improved insulin sensitivity and reduced hepatic glucose production.
   Example Baseline and Post-Treatment Values:
   Baseline fasting glucose: 140 mg/dL (hyperglycemic). Post-treatment: 110 mg/dL.
   Baseline HbA1c: 8.0%. ** Post-treatment: **6.9%.
2. Enhanced Insulin Secretion**
   Markers: Insulinogenic index, C-peptide levels.
   Therapeutic Thresholds:
   Insulinogenic Index: Increased by ≥30-50%, indicating improved early-phase insulin secretion.
   C-Peptide Levels: Sustained or increased, reflecting preserved or enhanced β-cell function.
   Example Baseline and Post-treatment Values:
   Insulinogenic index: Baseline 0.5; Post-treatment: 0.8 (higher values indicate better insulin secretion).
3. Weight Loss
   Marker: Percent Body Weight Reduction.
   Therapeutic Thresholds:
      Weight reduction of ≥5% is considered clinically significant for improving metabolic health, including liver-related outcomes.
      ≥10% weight loss correlates with substantial reductions in liver fat and histological improvement in NASH.
   Example Baseline and Post-Treatment Values:
      Baseline weight: 100 kg. Post-treatment: 90 kg (10% reduction).
4. Liver-specific Improvements Linked to These Effects**
   Markers: Liver enzymes (ALT/AST), liver fat (MRI-PDFF), and fibrosis scores (FibroScan®).
   Therapeutic Thresholds:
   ALT Reduction: ≥10-20 IU/L.
   Liver Fat (MRI-PDFF): Reduction by ≥30%.
   Fibrosis Improvement: ≥1 stage reduction in fibrosis score.
   Example Liver Improvements:
   Baseline ALT: 70 IU/L. Post-treatment: 50 IU/L.
   MRI-PDFF liver fat: Baseline 20%. Post-treatment: 14% (30% reduction).
5. Holistic Therapeutic Impact

The combination of improved glucose control, weight loss, and enhanced insulin secretion achieved through GLP-1 receptor activation not only slows disease progression but also reverses key features of NAFLD/NASH, such as hepatic steatosis, inflammation, and fibrosis.

These values align with clinical trial outcomes and established therapeutic targets for managing metabolic dysfunctions in NAFLD and NASH.

### ADVANTAGES OF THE PRESENT DISCLOSURE

The present disclosure provides a novel and effective approach to treat NAFLD and NASH through the administration of GLP-1 receptor agonists. This therapeutic strategy addresses the underlying metabolic and inflammatory components of the disease, offering a promising solution for a condition with limited treatment options. The combination of GLP-1 agonists with advanced delivery systems and synergistic therapeutic agents further enhances the efficacy of this approach.

### INDUSTRIAL APPLICATION

The present disclosure is beneficial to treat the following disease:
NAFLD/NASH;
Gut-Liver Axis Modulation; and
Preventive and Prophylactic Use.

### SPECIFIC EMBODIMENTS OF THE PRESENT DISCLOSURE

The present disclosure relates to a pharmaceutical composition for treating non-alcoholic fatty liver disease (NAFLD) or non-alcoholic steatohepatitis (NASH) comprising:
a GLP-1 receptor agonist, wherein the GLP-1 receptor agonist is selected from the group consisting of an SGLT-2 inhibitor, a PPAR agonist, a farnesoid X receptor (FXR) agonist, a fibroblast growth factor 21 (FGF21 ) analog, a statin and lipid lowering agent; and
a nanoparticle delivery agent.

Such pharmaceutical composition is disclosed, wherein the GLP-1 receptor agonist is administered at a dose to reduce liver enzymes including ALT and AST levels, markers of hepatic inflammation, and fibrosis.

Such pharmaceutical composition is disclosed, wherein the nanoparticle delivery agent is selected from the group consisting of lipid-based nanoparticles, polymeric nanoparticles, liposomes, exosomes-based delivery system, and inorganic nanoparticles.

Such pharmaceutical composition is disclosed, wherein the pharmaceutical composition is administered subcutaneously, intravenously, or orally.

Such pharmaceutical composition is disclosed, wherein the pharmaceutical composition has modified release properties.

Such pharmaceutical composition is disclosed, further comprises a bile acid sequestrant.

The present disclosure also relates to a GLP-1 receptor agonist, wherein the GLP-1 receptor agonist is selected from the group consisting of liraglutide, semaglutide, exenatide, dulaglutide, or a modified GLP-1 analog with an extended half-life and enhanced receptor affinity.

Such GLP-1 receptor agonist is disclosed, wherein the GLP-1 receptor agonist is delivered through a nano-particle delivery system.

Such GLP-1 receptor agonist is disclosed, wherein the GLP-1 receptor agonist is co-administered with additional agents, wherein the additional agents is selected from the group consisting of microbe modulator, probiotic, anti-fibrotic agent, and thyroid hormone receptor beta agonist.

Such GLP-1 receptor agonist is disclosed, wherein the GLP-1 receptor agonist is used for the treatment gastrointestinal side effects, metabolic-associated fatty liver disease, fibrosis, hepatitis, alcoholic liver disease (ALD), autoimmune hepatitis, hepatocellular carcinoma (HCC), hepatic fat accumulation, drug-induced liver injury, obesity-related liver disorders, hepatic encephalopathy, lipodystrophy-associated liver steatosis, hypertension, and polycystic ovary syndrome (PCOS).

The present disclosure also relates to a method of treating NAFLD and NASH with administration of the GLP-1 receptor agonist in a dosing regimen.

The present disclosure also relates to a kit for treating NAFLD or NASH, comprising:
the GLP-1 receptor agonist;
the delivery agent; and
instructional material regarding dosage and administration schedules.

1. A pharmaceutical composition for treating non-alcoholic fatty liver disease (NAFLD) or non-alcoholic steatohepatitis (NASH) comprising:
a GLP-1 receptor agonist, wherein the GLP-1 receptor agonist is selected from the group consisting of an SGLT-2 inhibitor, a PPAR agonist, a farnesoid X receptor (FXR) agonist, a fibroblast growth factor 21 (FGF21 ) analog, a statin and lipid lowering agent; and
a nanoparticle delivery agent.

2. The pharmaceutical composition as claimed in claim 1, wherein the GLP-1 receptor agonist is administered at a dose to reduce liver enzymes including ALT and AST levels, markers of hepatic inflammation, and fibrosis.

3. The pharmaceutical composition as claimed in claim 1, wherein the nanoparticle delivery agent is wherein the nanoparticle delivery agent is selected from the group consisting of lipid-based nanoparticles, polymeric nanoparticles, liposomes, exosomes-based delivery system, and inorganic nanoparticles.

4. The pharmaceutical composition as claimed in claim 1, wherein the pharmaceutical composition is administered subcutaneously, intravenously, or orally.

5. The pharmaceutical composition as claimed in claim 1, wherein the pharmaceutical composition has modified release properties.

6. The pharmaceutical composition as claimed in claim 1, further comprises a bile acid sequestrant.

7. A GLP-1 receptor agonist, wherein the GLP-1 receptor agonist is selected from the group consisting of liraglutide, semaglutide, exenatide, dulaglutide, or a modified GLP-1 analog with an extended half-life and enhanced receptor affinity.

8. The GLP-1 receptor agonist as claimed in claim 7, wherein the GLP-1 receptor agonist is delivered through a nano-particle delivery system.

9. The GLP-1 receptor agonist as claimed in claim 7, wherein the GLP-1 receptor agonist is co-administered with additional agents, wherein the additional agents is selected from the group consisting of microbe modulator, probiotic, anti-fibrotic agent, and thyroid hormone receptor beta agonist.

10. The GLP-1 receptor agonist as claimed in claim 7, wherein the GLP-1 receptor agonist is used for the treatment gastrointestinal side effects, metabolic-associated fatty liver disease, fibrosis, hepatitis, alcoholic liver disease (ALD), autoimmune hepatitis, hepatocellular carcinoma (HCC), hepatic fat accumulation, drug-induced liver injury, obesity-related liver disorders, hepatic encephalopathy, lipodystrophy-associated liver steatosis, hypertension, and polycystic ovary syndrome (PCOS).

11. A method of treating NAFLD and NASH with administration of the GLP-1 receptor agonist as claimed in claim 7 in a dosing regimen.

12. A kit for treating NAFLD or NASH, comprising:
the GLP-1 receptor agonist as claimed in claim 7;
the delivery agent; and
instructional material regarding dosage and administration schedules.
